(19) United States
(12) Patent Application Publication
(10) Pub. No.: US 2010/0324299 A1
(43) Pub. Date: Dec. 23, 2010

(54) METHOD FOR PURIFICATION OF PYRIDINE, AND METHOD FOR PRODUCTION OF CHLORINATED PYRIDINE

(75) Inventors: Naohiro Yoshikawa, Hyogo (JP); Seiji Bando, Hyogo (JP); Hirotsugu Konishi, Hyogo (JP); Chun Li, Hyogo (JP); Katsuhiko Yoshida, Hyogo (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: SUMITOMO SEIKA CHEMICALS CO., LTD., Kako-gun, Hyogo (JP)

(21) Appl. No.: 12/865,500

(22) PCT Filed: Mar. 23, 2009
(86) PCT No.: PCT/JP2009/055644

§ 371 Date: Sep. 7, 2010

(30) Foreign Application Priority Data
Mar. 31, 2008 (JP) 2008-94060

(51) Int. Cl. C07D 213/61; C07D 213/06

(52) U.S. Cl. 546/345; 546/353

(57) ABSTRACT

A method of purifying crude pyridine on an industrial scale at low cost and in a simple manner is provided. More specifically, a pyridine purification method including subjecting crude pyridine to an alkali treatment and then to distillation, or a pyridine purification method including adding an acid or water to crude pyridine, and subjecting the resulting mixture to an alkali treatment and then to distillation is provided. According to the purification method of the present invention, crude pyridine can be purified on an industrial scale at low cost and in a simple manner. Moreover, the pyridine purified by this method is useful as a starting material for various organic syntheses because of its high purity. For example, the pyridine can be reacted with chlorine to produce a chlorinated pyridine in a high yield.

### TECHNICAL FIELD

The present invention relates to a method of purifying pyridine, and a method of producing a chlorinated pyridine. More specifically, the present invention relates to a method of purifying pyridine comprising distilling crude pyridine, and a method of producing a chlorinated pyridine using the pyridine purified by this method.

### BACKGROUND ART

Pyridine is widely used, for example, as a starting material and solvent for organic synthesis of pharmaceuticals, pesticides, etc. Commercially available crude pyridine generally contains impurities such as aldehydes, alcohols, amines, etc. The use of such crude pyridine as a starting material for organic synthesis results in reduced yield and quality of desired reaction products.

Various methods have been proposed so far to purify pyridine. For example, Patent Document 1 discloses a method of purifying pyridine with a purity of 99% or higher by treatment with solid alkali in a vapor phase. Patent Document 2 discloses a method of purifying pyridine bases in such a manner that permanganate or dichromate is added and mixed into a solution containing a crude pyridine base or pyridine base, followed by allowing the mixture to stand at room temperature or at an elevated temperature; and then benzene is added, followed by azeotropic dehydration and then rectification. Patent Document 3 discloses a method of purifying pyridine by bringing it into contact with sodium borohydride, which is used as a reducing agent.

These methods, however, have various drawbacks. For example, the method of Patent Document 1 is not economical because a large amount of alkali is necessary for the production of a solid alkali phase, and a large amount of alkali waste is generated. Additionally, this method does not seem to be industrially preferable because obstruction of the alkali layer may occur due to the deliquescence of the solid alkali. The method of Patent Document 2 does not seem to be industrially advantageous because the operation is complicated, and the use of heavy metal salts results in increased waste liquid disposal costs. The method of Patent Document 3 does not seem to be industrially advantageous because sodium borohydride, which is expensive, is used.

### Patent Document 1: Japanese Unexamined Patent Publication No. 61-251662

### Patent Document 2: Japanese Unexamined Patent Publication No. 62-129269

### Patent Document 3: Japanese Unexamined Patent Publication No. 1-261368

### DISCLOSURE OF THE INVENTION

### Problems to be Solved by the Invention

An object of the present invention is to provide a method of purifying crude pyridine on an industrial scale at low cost and in a simple manner.

### Means for Solving the Problems

The present invention relates to a method of purifying pyridine comprising subjecting crude pyridine to an alkali treatment and then to distillation. The invention also relates to a method of purifying pyridine comprising adding an acid or water to crude pyridine, and subjecting the resulting mixture to an alkali treatment and then to distillation.

The invention further relates to a method of producing a chlorinated pyridine by reacting the pyridine purified by this method with chlorine.

The invention is described in detail below.

The purity of crude pyridine used in the method of purifying pyridine according to the present invention is 99% or higher and less than 100%, although not limited thereto. The crude pyridine to be used herein includes those obtained by known synthetic methods or commercial products; for example, crude pyridine containing imines (100 to 5000 ppm) or aldehydes (100 to 5000 ppm) as impurities, and also containing alcohols, amines, etc., are available.

The alkali treatment is specifically conducted by, for example, adding a predetermined amount of base to crude pyridine, followed by uniform stirring.

Examples of bases used for the alkali treatment include sodium hydroxide, sodium hydrogen carbonate, sodium acetate, sodium carbonate, potassium carbonate, potassium hydroxide, potassium acetate, calcium hydroxide, calcium carbonate, magnesium hydroxide, magnesium carbonate, lithium hydroxide, and the like. Among these, sodium hydroxide and potassium hydroxide are preferably used from an economic standpoint. These bases may be used singly or in combination of two or more. If necessary, these bases may be added to water and then added to the crude pyridine as a basic aqueous solution.

When a basic aqueous solution is used in the alkali treatment, the concentration of the solution is preferably 5 to 90 wt. %, and more preferably 10 to 70 wt. %. When the concentration of the solution is less than 5 wt. %, water tends to mix with the purified pyridine and thereby reduce purity, or the purification of the pyridine may be insufficient because of its water content. When the concentration is more than 90 wt. %, the reaction system tends to be inhomogeneous, reducing the effect of purifying the pyridine.

The amount of base used is preferably 0.01 to 20 parts by weight, and more preferably 0.02 to 3 parts by weight, based on 100 parts by weight of crude pyridine. When the amount of base used is less than 0.01 parts by weight, the purification of the pyridine may be insufficient; whereas, when the amount used is more than 20 parts by weight, an effect worth the amount used can hardly be obtained, which is not economical.

Moreover, when the alkali treatment is conducted after an acid is added to the crude pyridine, the base must be used in an amount greater than that necessary to neutralize the remaining acid. Accordingly, when an acid is added to the crude pyridine before the alkali treatment, it is preferable that the amount of base used to carry out the alkali treatment is equal to the amount that is used when no acid is added plus an amount of base that is sufficient for neutralizing the remaining acid. Although the amount of base used depends on the type and amount of acid used, for example, it is preferably 0.02 to 40 parts by weight, and more preferably 0.04 to 6 parts by weight, based on 100 parts by weight of crude pyridine.

The temperature of the alkali treatment is preferably −10° C. to 115° C., more preferably −10° C. to 90° C., and most preferably 10° C. to 70° C. At treatment temperatures less than −10° C., the purification of the pyridine may be insufficient; whereas at treatment temperatures more than 115° C., an effect worth the input energy can hardly be obtained, which is not economical.

The duration of the alkali treatment is, for example, 0.5 to 20 hours, and preferably 1 to 10 hours. For a treatment time that is less than 0.5 hours, the purification of the pyridine may be insufficient; whereas for a treatment time that is more than 20 hours, an effect worth the treatment time can hardly be obtained, which is not economical.

Although it is uncertain why pyridine can be purified by treating crude pyridine with alkali, followed by distillation, it is considered, for example, that aldehydes contained in the crude pyridine as impurities are condensed into high-boiling point compounds, facilitating the separation thereof from the pyridine during distillation.

In the method of purifying pyridine according to the invention, an acid or water may be added to the crude pyridine before the alkali treatment. The addition of an acid or water can further enhance the purity of the resulting purified pyridine.

Examples of acids include inorganic acids, such as sulfuric acid, hydrochloric acid, boric acid, nitric acid, phosphoric acid, and hydrobromic acid; and organic acids, such as formic acid, acetic acid, oxalic acid, citric acid, benzoic acid, methanesulfonic acid, and benzenesulfonic acid. Among these, sulfuric acid, hydrochloric acid, and phosphoric acid are preferably used from an economic standpoint. These acids may be used singly or in combination of two or more. If necessary, these acids may be added to water and then added to the crude pyridine as an acidic aqueous solution.

When an acidic aqueous solution is added to the crude pyridine, the concentration of the solution is preferably 0.5 to 100 wt. %, and more preferably 50 to 100 wt. %. When the concentration of the solution is less than 0.5 wt. %, water tends to be mixed into the purified pyridine to reduce the purity.

The amount of acid used is preferably 0.01 to 20 parts by weight, and more preferably 0.02 to 3 parts by weight, based on 100 parts by weight of crude pyridine. When the amount of acid used is less than 0.01 parts by weight, the purification of the pyridine may be insufficient; whereas when the amount is more than 20 parts by weight, an effect worth the amount used can hardly be obtained, which is not economical.

In the invention, the type of water used prior to the alkali treatment is not limited, and deionized water, distilled water, etc., are available.

The amount of water used is, for example, preferably 0.1 to 30 parts by weight, and more preferably 0.3 to 5 parts by weight, based on 100 parts by weight of crude pyridine. When the amount of water used is less than 0.1 parts by weight, the purification of the pyridine may be insufficient; whereas when the amount is more than 30 parts by weight, an effect worth the amount used can hardly be obtained, which is not economical.

In the invention, it is preferable that after an acid or water is added to the crude pyridine, the mixture is stirred before the alkali treatment.

The temperature at which an acid or water is added to the crude pyridine and stirred is generally −10° C. to 100° C., and preferably 10° C. to 70° C. At temperatures less than −10° C., the purification of the pyridine may be insufficient; whereas at temperatures more than 100° C., an effect worth the energy by heating can hardly be obtained, which is not economical. The stirring time is preferably 0.1 to 10 hours, and more preferably 0.5 to 3 hours.

Although it is uncertain why the purity of the resulting purified pyridine can be further enhanced by adding an acid or water to the crude pyridine prior to the alkali treatment, it is considered, for example, that the decomposition of imines contained in the crude pyridine as impurities is involved.

That is, it is considered that as a result of the addition of an acid or water to the crude pyridine, the imines are hydrolyzed into aldehydes, which are then treated with alkali and are thereby condensed into high-boiling point compounds, thereby facilitating the separation thereof from the pyridine during distillation.

The addition of an acid to the crude pyridine is preferred to the addition of water, because the incorporation of water into the resulting purified pyridine can be reduced, and various imines are expected to be decomposed.

In the method of purifying pyridine according the invention, the distillation temperature is generally 30° C. to 150° C., although it depends on the pressure. The number of trays in a distillation column is 1 to 100, for example. The reflux ratio is 50/1 to 1/1, for example.

Due to its high purity, pyridine purified in this manner is useful as a starting material for various organic syntheses. For example, this pyridine can be reacted with chlorine to produce a chlorinated pyridine in a high yield.

Examples of chlorinated pyridines include 2-chloropyridine, 2,6-dichloropyridine, and the like.

The method of reacting the pyridine with chlorine is not limited. For example, the pyridine and chlorine can be reacted in a liquid phase or gas phase by using a radical initiator or by irradiating with the light of a high-pressure mercury lamp or the like.

Particularly, from the standpoint of enhancing the efficiency of the chlorination reaction, the pyridine and chlorine are preferably reacted in a gas phase using water as a diluent under UV irradiation.

The amount of chlorine used is, for example, 0.1 to 3 mol per mol of pyridine, although it depends on the type of the desired chlorinated pyridine.

The amount of water used is 1 to 30 mol per mol of pyridine, for example.

The reaction temperature is 180° C. to 300° C., for example.

As the light source for generating UV light, for example, a high-pressure mercury lamp, ultra high-pressure mercury lamp, low-pressure mercury lamp, UV LED, or the like can be used.

The thus-obtained chlorinated pyridine can be isolated in such a manner that it is condensed by cooling, and a base (e.g., sodium hydroxide) is added thereto, followed by distillation.

### Effect of the Invention

According to the method of the present invention, crude pyridine comprising pyridine as a main ingredient can be purified on an industrial scale at low cost and in a simple manner. Since the amounts of impurities, such as imines and aldehydes, in crude pyridine are reduced in pyridine purified by this method, a chlorinated pyridine can be produced in a high yield using the pyridine purified by the method of the invention as a starting material.

### BEST MODE FOR CARRYING OUT THE INVENTION

### EXAMPLE 1

Crude pyridine (1000 g; purity: 99.53%, imine content: 1000 ppm, and aldehyde content: 2600 ppm) and 0.6 g of 48% aqueous sodium hydroxide solution were added to a 2000-mL four-neck flask equipped with a stirrer, condenser tube, thermometer, and dropping funnel, and stirred at 40° C. for 4 hours. Then, simple distillation was performed, thereby obtaining 986 g of purified pyridine. The purity of the obtained purified pyridine was 99.7% (water: 0%, imine content: 950 ppm, and aldehyde content: 60 ppm).

Subsequently, the purified pyridine was subjected to a photochlorination reaction. A high-pressure mercury lamp was attached to a 2480-mL glass reactor, and the photochlorination reaction of pyridine was carried out at a reaction temperature of 220° C. Two pipes for spraying an aqueous pyridine solution and two pipes for introducing chlorine were attached to the reactor wall so that they were alternately located at symmetrical positions, and the direction of the introduction of each introduced gas was concyclic and horizontal. The proportion of purified pyridine, chlorine, and water used in the reaction was 1:0.5:7.0 by molar ratio. A 38 wt. % aqueous pyridine solution was introduced through the pyridine spraying pipes at a rate of 1190 g/hr, while chlorine was introduced through the chlorine spraying pipes at 210 g/hr. Under these conditions, the residence time of the reactant gas was 8.1 seconds, and the reaction was carried out for 40 minutes. Thus, 114.9 g (1.0 mol) of 2-chloropyridine was obtained. The yield of the obtained 2-chloropyridine was 23.0% with respect to the purified pyridine.

### EXAMPLE 2

Crude pyridine (1000 g; purity: 99.53%, imine content: 1000 ppm, and aldehyde content: 2600 ppm) and 5.0 g of water were placed in a 2000-mL four-necked flask equipped with a stirrer, condenser tube, thermometer, and dropping funnel, and stirred at 40° C. for 1 hour. Then, 0.6 g of 48% aqueous sodium hydroxide solution was added and stirred at 40° C. for 4 hours. Subsequently, simple distillation was performed, thereby obtaining 979 g of purified pyridine. The purity of the obtained purified pyridine was 99.20% (water: 0.4%, imine content: 85 ppm, and aldehyde content: 60 ppm).

The obtained purified pyridine was subjected to a photochlorination reaction in the same manner as in Example 1. Thus, 118.8 g (1.04 mol) of 2-chloropyridine was obtained. The yield of the obtained 2-chloropyridine was 27.5% with respect to the purified pyridine.

### EXAMPLE 3

Crude pyridine (1000 g; purity: 99.53%, imine content:

1000 ppm, and aldehyde content: 2600 ppm) and 0.6 g of 98% aqueous sulfuric acid solution were added to a 2000-mL four-necked flask equipped with a stirrer, condenser tube, thermometer, and dropping funnel, and stirred at 40° C. for 1 hour. Then, 1.6 g of 48% aqueous sodium hydroxide solution was added and stirred at 40° C. for 4 hours. Subsequently, simple distillation was performed, thereby obtaining 986 g of purified pyridine. The purity of the obtained purified pyridine was 99.82% (water: 0%, imine content: 25 ppm, and aldehyde content: 60 ppm).

The obtained purified pyridine was subjected to a photochlorination reaction in the same manner as in Example 1. Thus, 128.5 g (1.13 mol) of 2-chloropyridine was obtained. The yield of the obtained 2-chloropyridine was 29.7% with respect to the purified pyridine.

### Comparative Example 1

In Example 1, the crude pyridine used for the production of the purified pyridine was used in place of the purified pyridine, and the photochlorination reaction of pyridine was carried out in the same manner as in Example 1. Thus, 77.1 g (0.68 mol) of 2-chloropyridine was obtained.

The yield of the obtained 2-chloropyridine was 16.9% with respect to the crude pyridine.

### Comparative Example 2

Crude pyridine (1000 g; purity: 99.53%, imine content: 1000 ppm, and aldehyde content: 2600 ppm) was placed in a 2000-mL four-necked flask equipped with a stirrer, condenser tube, thermometer, and dropping funnel. Simple distillation was performed under stirring, thereby obtaining 986 g of purified pyridine. The purity of the obtained purified pyridine was 99.60% (water: 0%, imine content: 900 ppm, and aldehyde content: 2600 ppm).

The obtained purified pyridine was subjected to a photochlorination reaction in the same manner as in Example 1. Thus, 79.9 g (0.70 mol) of 2-chloropyridine was obtained. The yield of the obtained 2-chloropyridine was 17.6% with respect to the purified pyridine.

### Comparative Example 3

Crude pyridine (1000 g; purity: 99.53%, imine content: 1000 ppm, and aldehyde content: 2600 ppm) and 0.6 g of 98% sulfuric acid were placed in a 2000-ml, four-necked flask equipped with a stirrer, condenser tube, thermometer, and dropping funnel, and stirred at 40° C. for 1 hour. Subsequently, simple distillation was performed, thereby obtaining 986 g of purified pyridine. The purity of the obtained purified pyridine was 99.60% (water: 0%, imine content: 25 ppm, and aldehyde content: 3300 ppm).

The obtained purified pyridine was subjected to a photochlorination reaction in the same manner as in Example 1. Thus, 81.8 g (0.72 mol) of 2-chloropyridine was obtained. The yield of the obtained 2-chloropyridine was 17.8% with respect to the purified pyridine.

### INDUSTRIAL APPLICABILITY

The present invention provides a method of purifying crude pyridine comprising pyridine as a main ingredient on an industrial scale at low cost and in a simple manner. Since the amounts of impurities, such as imines and aldehydes, in the crude pyridine are reduced in the pyridine purified by this method, a method of producing a chlorinated pyridine in a high yield can be provided by using the pyridine purified by the method of the invention as a starting material.

1. A method of purifying pyridine comprising subjecting crude pyridine to an alkali treatment and then to distillation.

2. A method of purifying pyridine comprising adding an acid or water to crude pyridine, and subjecting the resulting mixture to an alkali treatment and then to distillation.

3. The method according to claim 2, wherein the acid is sulfuric acid, hydrochloric acid, or phosphoric acid.

4. The method according to any one of claims 1 to 3, wherein the base used for the alkali treatment is sodium hydroxide or potassium hydroxide.

5. The method according to claims 1 to 3, wherein the temperature of the alkali treatment is −10° C. to 115° C.

6. A method of producing a chlorinated pyridine, the method comprising reacting the pyridine purified by the method according to any one of claims 1 to 3, with chlorine.

7. The method according to claim 4, wherein the temperature of the alkali treatment is −10° C. to 115° C.

8. A method of producing a chlorinated pyridine, the method comprising reacting the pyridine purified by the method according to claim 4, with chlorine.
